(19) United States
(12) Plant Patent Application Publication
(10) Pub. No.: US 2005/0138713 P1
(43) Pub. Date: Jun. 23, 2005

(54) Verbena plant named 'Sunmarisakura'

(50) VERBENA HYBRIDA; VERBENA

(76) Inventors: Kenichi Suzuki, Ibaraki (JP); Yasunori Yomo, Kawasaki (JP); Naoto Takamura, Omihachiman (JP)

Correspondence Address: CHRISTIE, PARKER & HALE, LLP, PO BOX 7068, PASADENA, CA, 91109-7068, US

(21) Appl. No.: 10/789,430

(22) Filed: Feb. 27, 2004

(30) Foreign Application Priority Data
Dec. 19, 2003 (JP) 16477

(51) Int. Cl. A01H 5/00

(52) U.S. Cl. PLT/308

(57) ABSTRACT

Disclosed herein is a Verbena plant having a decumbent growth habit and long stems. The plant forms flowers in clusters with a great profusion of blooms. The flowering duration is long and the entire plant remains in bloom for a considerable period of time. The flower size is large and the petal color of flowers is light purplish pink. The plant is highly tolerant to heat, pests and diseases, particularly powdery mildew, and is moderately resistant to cold. ‘SUNMARISAKURA’ does not freely set seed due to self-sterility.

### BOTANICAL/COMMERCIAL CLASSIFICATION

Verbena hybrida/Verbena Plant

### VARIETAL DENOMINATION

cv. ‘Summarisakura’

### BACKGROUND OF THE VARIETY

1) Parent;

‘Sunmarisa’ (U.S. Plant Pat. No. 11130)

2) Progress

The present a new distinct cultivar of Verbena was created by heavy ion beam irradiation on the Verbena hybrid variety called ‘Sunmarisa’. ‘Sunmarisa’ (U.S. Plant Pat. No. 11130) is our Verbena hybrid variety and grown at Omi R&D Center, Suntory Flowers Ltd.

In July 1998, 5Gy of ionic nitrogen (135 MeV) was irradiated onto 128 pieces of in-vitro axillary bud of ‘Sunmarisa’ using the Ring Cyclotron at The Institute of Physical and Chemical Research. Two weeks later, elongated buds were grown from cuttings. March 1999, 5 varieties were selected in view of self-sterility. These plants were propagated by cutting and then grown in pot and planter box on trial from May 1999 at the Omi R&D Center of Suntory Flowers Ltd. Additionally the plants were grown in pot and planter box on trial in 2000. Finally one plant was selected from these 5 varieties in view of the long flower duration. By November 2000, the botanical characteristics of the finally selected plant were examined. As a result, it was concluded that this new Verbena plant is distinguishable from any other variety, whose existence is known to us, and is uniform and stable in its characteristics. This new variety of Verbena plant was named ‘Sunmarisakura’.

The new variety of the present invention can be distinguished from its parent ‘Sunmarisa’ in its self-sterility. Also the new variety can be distinguished from the similar variety ‘Sunmaripi’ in the flower color and self-sterility.

In the following description, the color-coding is in accordance with The Horticultural Colour Chart of The Royal Horticultural Society, London, England (R.H.S. Colour Chart).

‘Sunmarisa’ was used as the parent when obtaining this new variety, ‘Sunmarisakura’. The botanical characteristics of ‘Sunmarisa’ are as follows when grown at the Omi R&D Center of Suntory Flowers Ltd.
Plant:
   Growth habit.—Decumbent.
   Plant width.—Approximately 65-71 cm.
   Plant height.—Approximately 8-9 cm.
Stem:
   Diameter.—Approximately 1.7-2.2 mm.
   Anthocyanin pigmentation.—Absent.
   Pubescence.—Normal.
   Branching.—A moderate level of branching is present.
   Subterranean stem.—Absent but when the stems contact the surface of soil, the nodes take root in the ground and the plant growth thereby spreads.
   Length of internodes.—Approximately 1.7-2.7 cm.
Leaf:
   Phyllotaxis.—Opposite.
   Shape.—Oblong-lanceolate.
   Depth of incision.—Shallow.
   Margin.—Serrate.
   Length.—Approximately 2.9-3.6 cm.
   Width.—Approximately 1.6-2.4 cm.
   Leaf apex.—Mucronate.
   Leaf base.—Petiolate.
   Color.—Dark olive green (R.H.S. 141A) on the upper surface.
   Pubescence.—Sparse.
   Petiole.—Present.
   Thickness of petiole.—Approximately 1.0-1.2 mm.
   Length of petiole.—Approximately 1.2-2.5 mm.
Flower:
   Shape of cluster.—Obovate.
   Length of cluster.—Approximately 2.3-4.4 cm.
   Diameter of cluster.—Approximately 4.0-5.8 cm.
   Facing direction.—Upward.
   Outward curvature of petal.—Flat.
   Diameter.—Approximately 1.8-1.9 cm.
   Height.—Approximately 2.1-2.2 cm.
   Color.—Light purplish pink (R.H.S. 62C) on the upper surface.
   Eye color.—Pale yellow (R.H.S. lD).
   Variegation on petal.—Absent.
   Color presentation.—Substantially even.
   Color intensity.—Absent.
   Overlapping of petals.—Separate.
   Incision of petal.—Present.
   Number of petals.—5.
   Sepal.—Tubular in configuration.
   Length of calyx.—Approximately 1.2-1.4 cm.
   Anthocyanin pigmentation of calyx limb.—Absent.
   Shape of pistil.—Two lobes.
   Color of anther.—Yellowish green.
   Diameter of peduncle.—Medium.
   Length of peduncle.—Medium.
   Number of flowers.—Many.
   Flower bearing.—In a cluster.
   Reproductive organs.—1 pistil and 4 stamens.
   Pollen.—Brilliant greenish-yellow (R.H.S. 6C) in coloration.
   Flower fragrance.—Absent.
   Flowering time.—Early.
   Flowering duration.—Long. When planted during March, the plant commonly blossoms from April to November. A bloom cluster commonly is present for approximately 2 to 3 weeks, and an individual bloom within the cluster commonly lasts for approximately 7 to 10 days on the plant. The plant is highly tolerant to heat, and exhibits a high resistance to diseases, particularly powdery mildew, and a high resistance to pests.

‘Sunmaripi’ was used as a comparison for this new variety ‘Sunmarisakura’. The botanical characteristics of the ‘Sunmaripi’ are as follows when grown at when grown at the Omi R&D Center of Suntory Flowers Ltd.
Plant:
   Growth habit.—Decumbent.
   Plant width.—Approximately 60 cm.
   Plant height.—Approximately 20 cm.
Stem:
   Diameter.—Approximately 2.5 mm.
   Anthocyanin pigmentation.—Present.
   Pubescence.—Normal.
   Branching.—A moderate level of branching is present.
   Subterranean stem.—Absent but when the stems contact the surface of soil, the nodes take root in the ground and the plant growth thereby spreads.
   Length of internode.—Approximately 4.5 cm.
Leaf:
   Phyllotaxis.—Opposite.
   Shape.—Oblong-lanceolate.
   Depth of incision.—Shallow.
   Margin.—Serrate.
   Length.—Approximately 3.5 cm.
   Width.—Approximately 2.2 cm.
   Leaf apex.—Mucronate.
   Leaf base.—Petiolate.
   Color.—Grayish olive green (R.H.S. 147A) on the upper surface.
   Pubescence.—Sparse.
   Petiole.—Present.
   Thickness of petiole.—Approximately 1.0 mm.
   Length of petiole.—Approximately 2.0 mm.
Flower:
   Shape of cluster.—Obovate.
   Length of cluster.—Approximately 3.2 cm.
   Diameter of cluster.—Approximately 5.2 cm.
   Facing direction.—Upward.
   Outward curvature of petal.—Flat.
   Diameter.—Approximately 1.7 cm.
   Height.—Approximately 2.0 cm.
   Color.—Deep purplish pink (R.H.S. 77B) on the upper surface.
   Eye color.—Pale yellow (R.H.S. 11D).
   Variegation on petal.—Absent.
   Color presentation.—Substantially even.
   Color intensity.—Absent.
   Overlapping of petals.—Separate.
   Incision of petal.—Present.
   Number of petals.—5.
   Sepal.—Tubular in configuration.
   Length of calyx.—Approximately 1.0 cm.
   Anthocyanin pigmentation of calyx limb.—Present.
   Shape of pistil.—Two lobes.
   Color of anther.—Yellowish green.
   Diameter of peduncle.—Medium.
   Length of peduncle.—Medium.
   Number of flowers.—Many.
   Flower bearing.—In a cluster.
   Reproductive organs.—1 pistil and 4 stamens.
   Pollen.—Brilliant greenish-yellow (R.H.S. 6C) in coloration.
   Flower fragrance.—Absent.
   Flowering time.—Early.
   Flowering duration.—Long. When planted during March, the plant commonly blossoms from April to November. A bloom cluster commonly is present for approximately 2 to 3 weeks, and an individual bloom within the cluster commonly lasts for approximately 7 to 10 days on the plant. The plant is highly tolerant to heat, and exhibits a high resistance to diseases, particularly.powdery mildew, and a high resistance to pests.

### SUMMARY OF THE VARIETY

This new variety of Verbena plant has a broad spreading growth habit and long stems. The plant is well branched and abundantly forms flowers in a cluster. The blooms are present in profusion. The blooming period is April to November and flowering duration is long. The entire plant remains in bloom for an extended period of time. The flower size is large and the petal coloration of the flowers is light purplish pink. The plant is highly tolerant to heat, exhibits a high resistance to pests and diseases, particularly powdery mildew. The- plant has very low self-fertility

### BRIEF DESCRIPTION OF THE DRAWINGS

FIG. 1 depicts a side view of the ‘Sunmarisakura’ plant of the present invention.

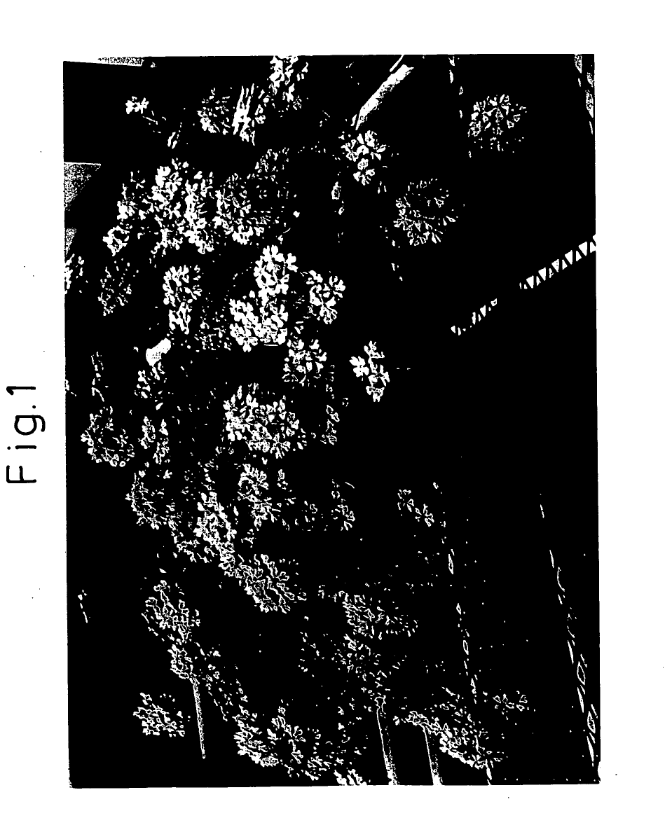

FIG. 2 depicts a close-up view of the flowers of the ‘Sunmarisakura’ plant of the present invention.

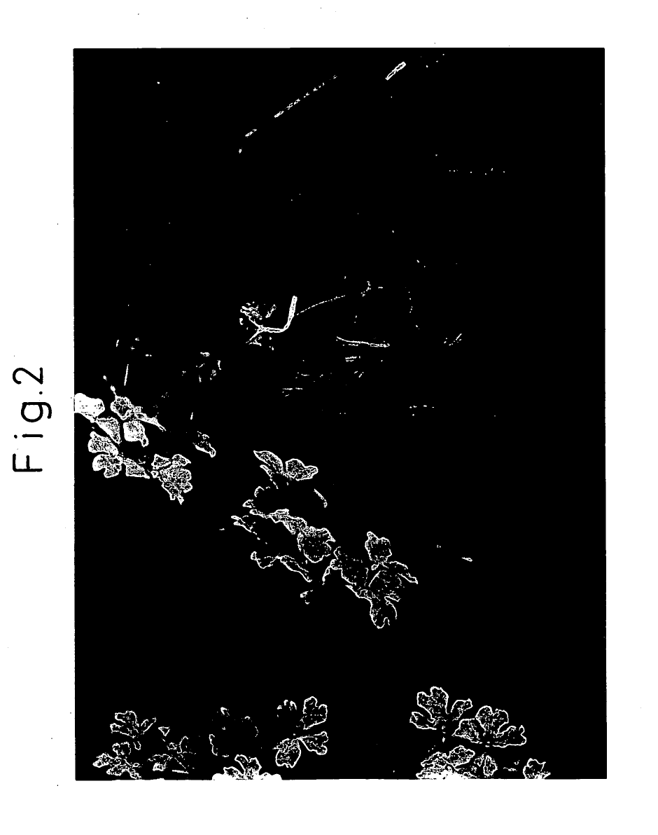

### DESCRIPTION OF THE VARIETY

The botanical characteristics of the new and distinct variety of Verbena plant, ‘Sunmarisakura’ are as follows when grown at the Omi R&D Center of Suntory Flowers Ltd.
Plant:
   Growth habit.—Decumbent.
   Plant width.—Approximately 65-71 cm.
   Plant height.—Approximately 8-9 cm.
Stem:
   Diameter.—Approximately 1.7-2.2 mm.
   Anthocyanin pigmentation.—Absent.
   Pubescence.—Normal.
   Branching.—A moderate level of branching is present.
   Subterranean stem.—Absent but when the stems contact the surface of soil, the nodes take root in the ground and the plant growth thereby spreads.
   Length of internode.—Approximately 1.7-2.7 cm.
Leaf:
   Phyllotaxis.—Opposite.
   Shape.—Oblong-lanceolate.
   Depth of incision.—Shallow.
   Margin.—Serrate.
   Length.—Approximately 2.9-3.6 cm.
   Width.—Approximately 1.6-2.4 cm.
   Leaf apex.—Mucronate.
   Leaf base.—Petiolate.
   Color.—Dark olive green (R.H.S. 141A) on the upper surface.
   Pubescence.—Sparse.
   Petiole.—Present.
   Thickness of petiole.—Approximately 1.0-1.2 mm.
   Length of petiole.—Approximately 1.2-2.5 mm.
Flower:
   Shape of cluster.—Obovate.
   Length of cluster.—Approximately 2.3-4.4 cm.
   Diameter of cluster.—Approximately 4.0-5.8 cm.
   Facing direction.—Upward.
   Outward curvature of petal.—Flat.
   Diameter.—Approximately 1.8-1.9 cm.
   Height.—Approximately 2.1-2.2 cm.
   Color.—Light purplish pink (R.H.S. 62C) on the upper surface.
   Eye color.—Pale yellow (R.H.S. 11D).
   Eye size.—Small.
   Variegation on petal.—Absent.
   Color presentation.—Substantially even.
   Color intensity.—Absent.
   Overlapping of petals.—Separate.
   Incision of petal.—Present.
   Number of petals.—5.
   Sepal.—Tubular in configuration.
   Length of calyx.—Approximately 1.2-1.4 cm.
   Anthocyanin pigmentation of calyx limb.—Absent.
   Shape of pistil.—Two lobes.
   Color of anther.—Yellowish green.
   Diameter of peduncle.—Medium.
   Length of peduncle.—Medium.
   Number of flowers.—Many.
   Flower bearing.—In a cluster.
   Reproductive organs.—1 pistil and 4 stamens.
   Pollen.—Brilliant greenish-yellow (R.H.S. 6C) in coloration.
   Flower fragrance.—Absent.
   Flowering time.—Early.
   Flowering duration.—Long. When planted during March, the plant commonly blossoms from April to November. A bloom cluster commonly is present for approximately 2 to 3 weeks, and an individual bloom within the cluster commonly lasts for approximately 7 to 10 days on the plant. The plant is highly tolerant to heat, and exhibits a high resistance to diseases, particularly powdery mildew, and a high resistance to pests.

This new variety of Verbena plant is most suitable for flower bedding and potting, particularly in planters, and is further excellent for use as a hanging basket.

The plant of this new variety, ‘Sunmarisakura’, is presently planted and maintained at the Omi R&D Center of Suntory Flowers Ltd., 863-1 Aza-Iketani, Omori-cho, Yokaichi-shi, Shiga-ken, Japan.

1. A new and distinct variety of Verbena hydrida plant having the following combination of characteristics:
(a) exhibits a decumbent growth habit with long stems,
(b) forms in abundance clusters of attractive light purplish pink blossoms that remain on the plant for an extended period of time,
(c) exhibits good tolerance to heat, pests and diseases, and
(d) has very low self-fertility.
